(19) United States
(12) Patent Application Publication
(10) Pub. No.: US 2017/0043100 A1
(43) Pub. Date: Feb. 16, 2017

(54) ECHOGENIC PATTERN AND MEDICAL ARTICLES INCLUDING SAME

(71) Applicants: Access Scientific, LLC, San Diego, CA (US); Cadence Science, Inc., Cranston, RI (US)

(72) Inventors: Tu Nguyen, Escondido, CA (US); David Joseph Bealka, East Freetown, MA (US)

(21) Appl. No.: 15/234,841

(22) Filed: Aug. 11, 2016

Related U.S. Application Data
(60) Provisional application No. 62/203,855, filed on Aug. 11, 2015.

(51) Int. Cl. A61M 5/32; A61B 34/20

(52) U.S. Cl. CPC A61M 5/329; A61B 34/20; A61B 2034/2063

(57) ABSTRACT

A medical article, such as a needle, includes an echogenic portion. The echogenic portion includes one or more patterns. The one or more patterns can include closely spaced surface features which provide enhanced echogenicity. The surface features can include grooves, recesses, channels, pits, pores, or the like. The grooves can be arranged in a zigzag shape in a needle body of the medical article. The zigzag shape can extend circumferentially around the needle body, that is, the grooves can extend perpendicularly to a longitudinal axis of the needle body. The zigzag shape provides enhanced echogenicity regardless of the section of the needle body underlying the ultrasound beam in use.

### CROSS-REFERENCE TO RELATED APPLICATIONS

This application claims priority to and the benefit of U.S. Provisional Patent Application Ser. No. 62/203,855, filed Aug. 11, 2015, the entirety of which is hereby expressly incorporated by reference herein.

### BACKGROUND

Field

The present disclosure is generally directed to medical articles (for example, needles, catheters, cannulas, sheaths, etc.) including features that provide enhanced ultrasound visibility during introduction and/or delivery into a body space, such as, for example, an artery, vein, vessel, body cavity, or drainage site.

Description of the Related Art

Various medical devices, for example, needles, catheters, cannulas, sheaths, etc., are often introduced into a patient, for example, in an artery, vein, body cavity, or drainage site, to deliver fluids to or withdraw fluids from the patient. In some cases, a needle can be introduced into a patient, for example, into a blood vessel, under the guidance of ultrasound. The ultrasound permits a physician or other medical personnel to view the position of the needle relative to the patient's vasculature and surrounding tissue. Ultrasound guidance allows the physician to monitor the position of the needle and ensure proper insertion of the needle in the patient during the procedure. In some cases, a physician may need to relocate the needle and interact with the surrounding tissue during insertion and/or after insertion during use.

### SUMMARY

The medical devices, such as needles, described herein advantageously provide for improved echogenicity.

In some embodiments, a medical article includes a needle body having a proximal end, a distal end, and a lumen extending therethrough. The needle body includes an echogenic portion. The echogenic portion includes at least one groove having a zigzag configuration.

In some embodiments, a medical article includes a needle body having a proximal end, a distal end, and a lumen extending therethrough. The distal end includes an echogenic portion having a plurality of grooves. Each of the plurality of grooves comprises a series of alternating proximal facing peaks and distal facing peaks.

In some embodiments, an access device includes a needle having an elongated needle body and a needle hub from which the needle body extends. The needle body includes a proximal end, a distal end, a lumen extending therethrough, and an echogenic portion. The echogenic portion is formed by at least one groove shaped in a zigzag configuration. The access device further includes a dilator slideably disposed on the needle body. The dilator includes a dilator hub and an elongated dilator shaft that extends from the dilator hub. The access device further includes a sheath slideably disposed on the dilator shaft. The sheath includes a tubular sheath body and a sheath hub coupled with the tubular sheath body.

In some embodiments, a medical article includes a needle body having a proximal end, a distal end, and a lumen extending therethrough. The needle body includes an echogenic portion including one or more surface features forming one or more patterns. The surface features can be one or more grooves. In some embodiments, each of the one or more surface features forms a zigzag pattern. In some embodiments, the zigzag patterns circumferentially encircle the needle body.

In some embodiments, the echogenic portion includes a plurality of grooves, each of the plurality of grooves having a zigzag configuration and circumferentially encircling the needle body. In some such embodiments, the echogenic portion includes ten grooves. The grooves can be spaced apart by about 0.031 inches. Each groove can have a series of alternating proximal facing peaks and distal facing peaks, and the proximal facing peaks of a first of the plurality of grooves can be at least substantially circumferentially aligned with the distal facing peaks of a second of the plurality of grooves positioned adjacent and proximal to the first of the plurality of grooves. In some embodiments, each groove is about 0.0025 inches to about 0.0033 inches wide. In some embodiments, each groove is about 0.0015 inches to about 0.003 inches deep.

The distal end of the needle body can include a beveled tip. In some embodiments, the distal end includes a dual-beveled tip having a first bevel and a second bevel. In some embodiments, the needle includes at least one opening in a wall of the needle body. In some embodiments, the needle body includes a laser-etched right-hand spiral positioned at or near the proximal end and configured to engage a needle hub.

A method of forming an echogenic needle body can include forming grooves in a surface of the needle body and grinding a distal end of the needle body to form a beveled tip. In some embodiments, the grooves are laser etched into the surface of the needle body. The grooves can be formed in a zigzag pattern circumferentially encircling the needle body.

A method of using a medical article that includes at least a single groove in a zigzag pattern encircling a needle body proximate to a distal end can included inserting the medical article into a vessel of a patient, placing an ultrasound transducer in proximity to the patient and above the medical article, transmitting ultrasound energy in a direction towards the zigzag pattern on the medical article, and sensing a return signal reflected off the zigzag pattern to ensure proper insertion of the medical article into the vessel.

These and other aspects of the present disclosure will become readily apparent to those skilled in the art from the following detailed description, which refers to the attached figures. The invention is not limited, however, to the particular embodiments that are disclosed.

### BRIEF DESCRIPTION OF THE DRAWINGS

These and other features, aspects, and advantages of the present disclosure are described below with reference to the drawings of certain embodiments, which are intended to illustrate certain embodiments and not to limit the disclosure.

FIG. 1A is a side view of an example embodiment of a medical article comprising an echogenic portion.

FIG. 1B is an enlarged side view of a distal portion of the medical article depicted in FIG. 1A and shows the echogenic portion having a plurality of grooves in a zigzag pattern.

FIG. 1C is a perspective cross-section view through the echogenic portion of a needle body of the medical article from FIG. 1B showing the plurality of grooves in a surface of the needle body.

FIG. 1D is a cross-section view through the echogenic portion of the needle body of the medical article from FIG. 1B showing the plurality of grooves in the surface of the needle body.

FIG. 2 is an enlarged perspective view of the distal portion of the medical article of FIGS. 1A and 1B.

FIG. 3A is a side view similar to FIG. 1A except the medical article has been rotated counter-clockwise 90° degrees and includes an opening or fenestration.

FIG. 3B is an enlarged view of a portion of the medical article depicted in FIG. 3A showing the opening or fenestration.

FIG. 4 illustrates an exemplary embodiment of a medical access device that can be inserted into a blood vessel (e.g., a vein or an artery) and that can include the needle body depicted in FIGS. 1A and 1B.

FIGS. 5A, 5B, 5C, 5D, 6A, 6B, 6C, and 6D show comparative echo-photos of the medical article of FIGS. 1A-3B and a first needle having another echogenic design.

### DETAILED DESCRIPTION

Embodiments of the invention will now be described with reference to the accompanying figures, wherein like numerals refer to like elements throughout the following description and drawings. Although several embodiments, examples and illustrations are disclosed below, it will be understood by those of ordinary skill in the art that the present disclosure extends beyond the specifically disclosed embodiments, examples and illustrations and can include other uses and obvious modifications and equivalents thereof. The terminology used in the description presented herein is not intended to be interpreted in any limited or restrictive manner simply because it is being used in conjunction with a detailed description of certain specific embodiments of the disclosure. In addition, embodiments of the disclosure can comprise several novel features and no single feature is solely responsible for its desirable attributes or is essential to practicing the present disclosure.

In various circumstances a physician may wish to introduce a needle, catheter, or sheath into a space within a patient's body, for example, a blood vessel or drainage site, to introduce fluids to the space or remove fluids from the space. During or after insertion of the needle into the patient, the physician may wish to ensure proper insertion of the needle into the patient's vessel. Improper insertion can inhibit the success of the procedure being performed. Additionally, when a needle is inserted incorrectly or when several needle insertions are required, this can cause the patient discomfort that may otherwise be avoided with the use of ultrasound guidance.

The present disclosure provides medical articles, such as needles, having one or more echogenic features to improve visibility under ultrasound. In some embodiments, needles according to the present disclosure can be included in medical access devices for the introduction or delivery of a medical article, such as a catheter, cannula, or sheath, into a space, such as a blood vessel or drainage site. In some embodiments, needles according to the present disclosure can be used for biopsy or tissue sampling purposes. While the exemplary embodiments are described as a needle, in some embodiments, other medical articles, such as dilators or sheaths, can include echogenic features as described herein.

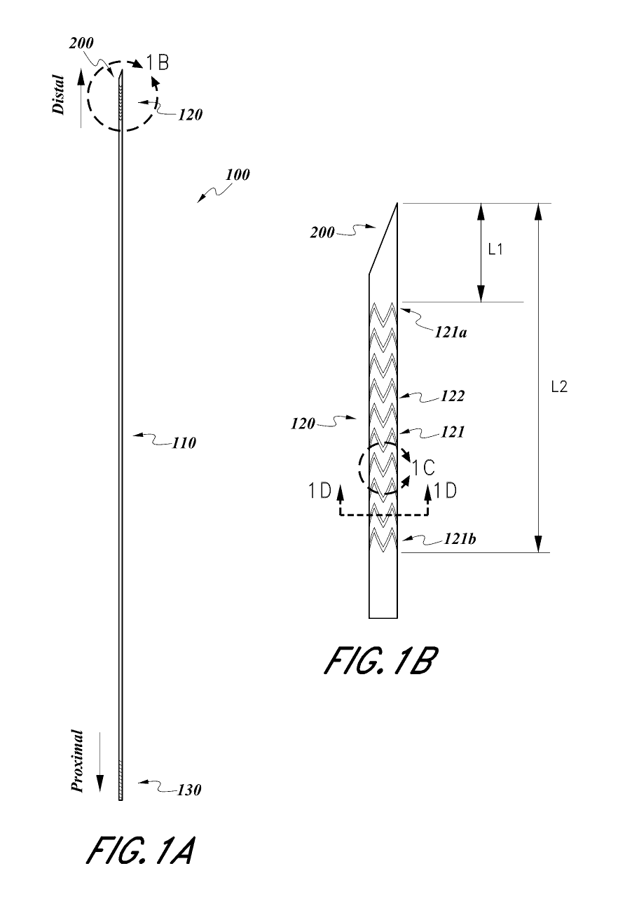

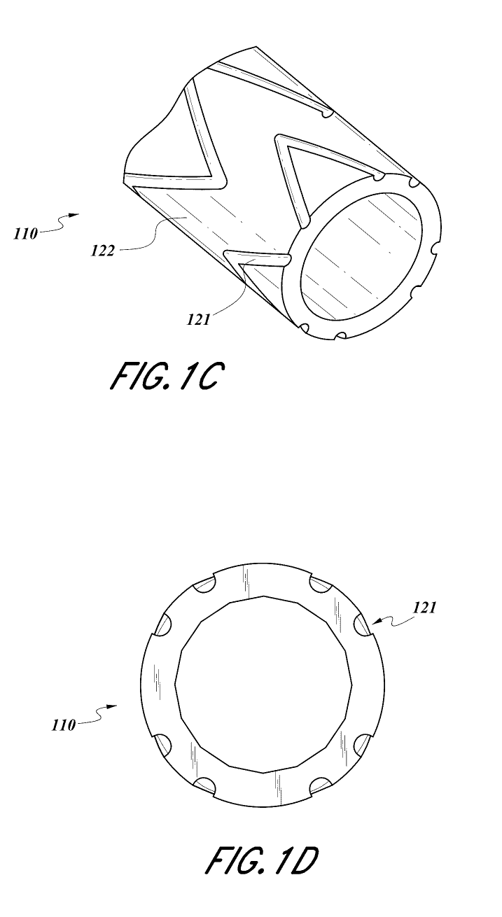

FIG. 1A illustrates an example embodiment of a medical article 100 configured as a needle for insertion into a body space, such as a blood vessel (e.g., a vein or an artery), while under the guidance of ultrasound visualization. Although various embodiments are described herein in the context of vascular access, the needles described herein can also can be used to access other locations within a patient's body (e.g., a drainage site) and/or for other purposes (e.g., for draining an abscess).

As shown in FIG. 1A, the medical article 100 includes a needle body 110 having an elongated tubular shape and a lumen extending therethrough from a proximal end to a distal end of the needle body 110. As used herein, the term distal refers to a direction away from a user of the medical article 100, while the term proximal refers to a direction approaching a user. The needle body 110 can have a shaped distal tip 200 at the distal end. The distal tip 200 can be configured to puncture tissues (e.g., skin, muscle, a blood vessel wall, etc.) of the patient. In certain embodiments in which the medical article 100 is configured as a dilator, the distal tip of the dilator can expand an opening in the tissue of the patient.

In the illustrated embodiment, the medical article 100 has a generally circular lumen and a generally circular exterior surface. In other embodiments, however, the needle body 110 can have other lumen and exterior shapes (such as, for example, but without limitation, an oval cross-sectional shape, tapering cross-sectional shapes, or other suitable shapes). In certain embodiments, the needle body 110 has a sufficiently long length to access a targeted subcutaneous body space and has a sufficient gauge size to withstand the insertion forces when accessing the body space without causing undue trauma. For many applications, the needle body 110 can have a length in the range of about 3 to about 20 cm, for example, about 15 cm. The size of the needle preferably is 18 gauge or smaller, and more preferably between 18-28 gauge, and most preferably between 18 and 26 gauge for micro-puncture applications (e.g., peripheral IVs). For applications with a neonate, the length and gauge of the needle body 110 should be significantly shorter and smaller, for example preferably between 3-4 cm and between 26-28 gauge.

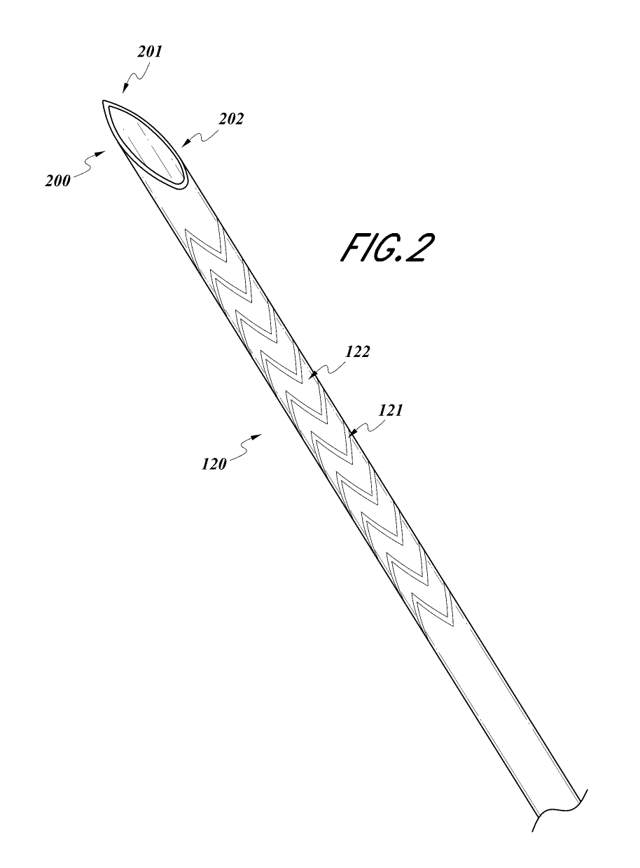

As shown in greater detail in FIGS. 1B, 1C, and 2, the needle body 110 has one or more echogenic portions 120. In certain embodiments, the one or more echogenic portions are proximate to the distal end. In certain embodiments, the echogenic portion 120 comprises one or more surface features such as grooves, recesses, channels, pits, pores, or the like. In certain embodiments, a wall thickness of the needle body 110 in the one or more echogenic portions 120 varies at the locations of the one or more surface features. In certain embodiments, the variations in wall thickness of the needle body 110 enhance the echogenicity of the needle body 110. The one or more surface features can be disposed on the outside surface diameter and/or inside surface diameter of the needle body 110. In certain embodiments, the one or more surface features have a roughened surface compared to a relatively smoother surface of the bare needle body 110.

In certain embodiments, the one or more surface features form one or more patterns in the one or more echogenic portions 120. The one or more patterns comprise closely spaced surface features which provide enhanced echogenicity. In the illustrated embodiment, the pattern comprises a groove 121 or a plurality or series of grooves 121 in the outer surface of the needle body 110. The groove 121 may be continuous or broken along its length in certain embodiments. In certain embodiments, a width and/or depth of the groove 121 is consistent along a length of the groove 121.

In certain embodiments, the groove 121 generally extends circumferentially (i.e., perpendicularly to a longitudinal axis of the needle body 110) around an entire circumference of the needle body 110. In certain embodiments, the groove 121 extends about only a portion of the circumference of the needle body 110 while still providing improved echogenicity. As the groove 121 extends circumferentially, the groove 121 may form a shape or pattern. The pattern can be symmetrical, asymmetrical along one or more of the x, y, and z axes. In certain embodiments, the groove 121 forms a sinusoidal, zigzag, sawtooth, chevron, or other annular configuration or any combination thereof. In certain embodiments, the groove 121 forms a caret shape or series of caret shapes. The caret or carets can be oriented in various directions and configurations. Shapes or patterns having relatively sharp and/or angular features can provide improved echogenicity.

In the illustrated embodiment, the pattern includes a plurality of grooves 121. Preferably a density of the grooves 121 on the needle body 110 is selected so that a combined surface area of the grooves 121 is sufficient to provide improved echogenicity. For example, a ratio of the combined surface area of the grooves 121 compared to a surface area of bare (e.g., non-grooved) needle body 110 in the echogenic portion 120 can be in the range of about 25% to about 80%, for example, about 50%. In certain embodiments, a contrast between the roughened surface of the grooves 121 or other surface features and the relatively smooth surface of the bare needle body 110 in the echogenic portion 120 under ultrasound enhances echogenicity. The plurality of grooves 121 may be spread evenly across the circumference of the needle body 110 or concentrated in one or more regions of the needle body 110. Each groove 121 is offset 122 from an adjacent groove 121 along the longitudinal axis of the needle body 100. Of course the offset or distance 122 between grooves 121 need not be equal and may vary between grooves 121.

FIG. 1C is a perspective cross-section view through the echogenic portion of the needle body 110 of the medical article from FIG. 1B showing the plurality of grooves 121 in a surface of the needle body 110. In the illustrated embodiment, the grooves 121 have a zigzag shape or configuration and extend circumferentially around the entire circumference of the needle body 110. As shown, each zigzag shaped groove 121 has a series of alternating proximal facing peaks and distal facing peaks connected by lines. The peaks may be sharp, rounded, or have another shape. In certain embodiments, the lines connecting the peaks of the zigzag shape are straight. Of course the lines need not be straight and may have one or more curves along their lengths. In embodiments where the groove 121 is continuous, the lines intersect at each peak. The angle between intersecting lines can be 90 degrees, obtuse, or acute. In certain embodiments, the angle is 51 degrees. In certain embodiments, the angle between intersecting lines is determined by the number of zigzags around the circumference of the needle body 110. For an embodiment where the angle between intersecting lines is 51 degrees and the size of the needle body 110 is 21 gauge, eight zigzags can be employed around the circumference. In certain embodiments where the size of the needle body 110 is 4 FR or 5 FR, eight zigzags can be employed. In certain embodiments where the size of the needle body 110 is 4 FR, the angle between intersecting lines is about 48 degrees. Of course more or less zigzags can be employed depending on the size of the needle body 110 and the desired angle between intersecting lines.

In the illustrated embodiment, the grooves 121 are aligned with one another such that proximal facing peaks of the groove 121 are longitudinally aligned with proximal facing peaks of the adjacent groove 121 and distal facing peaks of the groove 121 are longitudinally aligned with distal facing peaks of the adjacent groove 121. Of course the peaks of adjacent or offset grooves 121 need not be aligned along the longitudinal axis. In certain embodiments, the groove 121 is slightly rotated about the longitudinal axis relative to the adjacent groove 121 so that the peaks of adjacent grooves 121 are not exactly aligned.

Adjacent grooves 121 can be evenly spaced or have the same pitch between adjacent grooves, as shown in FIGS. 1B, 1C, and 2. However, in other embodiments, the spacing between adjacent grooves 121 varies. As shown, the adjacent grooves 121 are spaced apart along the longitudinal axis such that the proximal facing peaks of the groove 121 are at least substantially circumferentially aligned with the distal facing peaks of the groove 121. In certain embodiments, the adjacent grooves 121 are spaced close enough so that the peaks of the groove 121 are substantially aligned with the lines connecting the peaks of the adjacent groove 121. In certain embodiments, the adjacent grooves 121 are spaced apart a sufficient distance 122 so that the peaks of the groove 121 are not substantially aligned with the peaks or the lines of the adjacent groove 121. In some embodiments, each groove 121 has a pitch of about 0.031 in. In some such embodiments, a space 122 between adjacent grooves 121 can have a length of about 0.031 in.

The grooves 121 are distributed over a sufficient portion of the needle body 110 to ensure increased echogenic properties. In the illustrated embodiment, the needle body 110 comprises a total of 10 grooves 121, although more or fewer grooves 121 are also possible. In some embodiments, a distance L1 (shown in FIG. 1B) from the distal end of the needle body 110 to a distal-most groove 121A can be in the range of about 0.088 in. to about 0.158 in., for example 0.123 in., and a distance L2 from the distal end of the needle body 110 to a proximal-most groove 121B can be in the range of about 0.403 in. to about 0.485 in, for example 0.473 in.

FIG. 1D is a cross-section view through the echogenic portion of the needle body 110 of the medical article from FIG. 1B showing the plurality of grooves 121 in the surface of the needle body 110. A cross-section of each groove 121 may have a truncated circular shape, a U-shape, a V-shape, or other shape. Each of the grooves 121 illustrated in FIG. 1D has a U-shaped cross-section. In some embodiments, each groove 121 is about 0.0025 in. to about 0.0033 in. wide, for example 0.003 in. wide, and about 0.0015 in. to about 0.003 in. deep. Increasing the depth of the groove 121 while maintaining a sufficient thickness between a bottom surface of the groove 121 and the inside diameter of the needle body 110 improves echogenicity. The thickness between the bottom surface of the groove 121 and the inside diameter of the needle body 110 is selected to prevent burning through the needle body 110 during fabrication.

The grooves 121 can be formed by various methods. For example, the grooves 121 can be etched, e.g., laser etched, into the surface of the needle body 110. In other embodiments, the grooves 121 may be formed by photo-etching, e-beam, grinding, or wat-jet cutting. In some embodiments, the pattern may be formed by a surface material applied to the needle surface instead of or in addition to grooves 121 formed in the needle body 110. In certain embodiments, a method of manufacturing a medical article according to the present disclosure includes cutting a needle or other tubing to a desired length. Forming the echogenic portion 120, for example, by forming grooves 121 in the surface of the tubing. The grooves 121 can be formed by, for example by laser etching, grinding, wat-jet cutting, or other methods, or applying a surface material or overmolding to the surface of the tubing in the desired pattern. The distal tip 200 can be formed by grinding either before or after forming the echogenic portion 120. In some embodiments, the method of manufacturing further includes washing the medical article after forming the echogenic portion 120 and/or after forming the distal tip 200. Washing can remove inclusions, burns, or foreign material such as dirt, grease, oil, coatings or processing solutions.

The illustrated pattern advantageously provides a greater surface density of echogenic features on the needle body 110, and therefore a reduced area of bare needle in the echogenic portion 120, which improves visibility of the needle notwithstanding what section of the needle body 110 lies under the plane of the ultrasound beam in use.

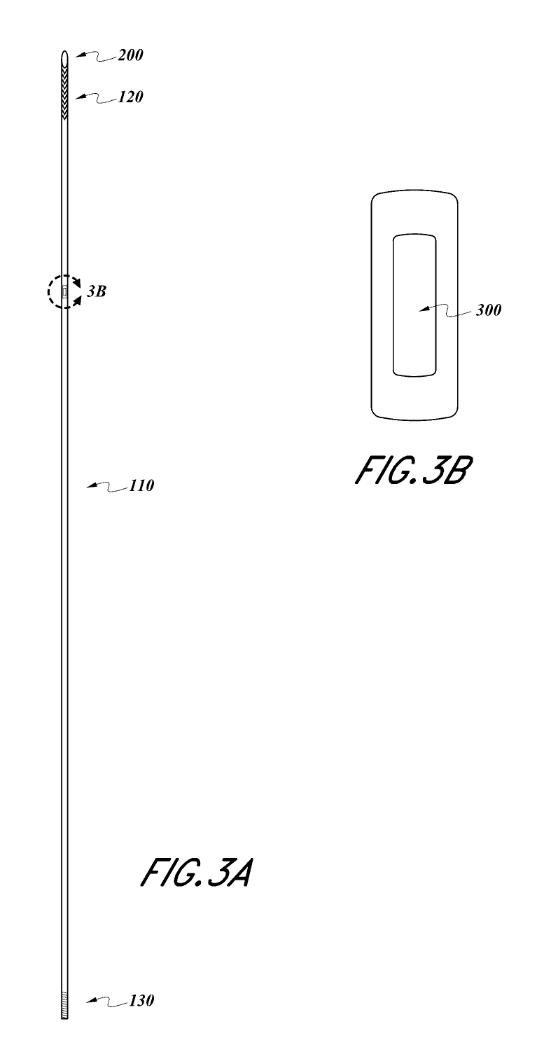

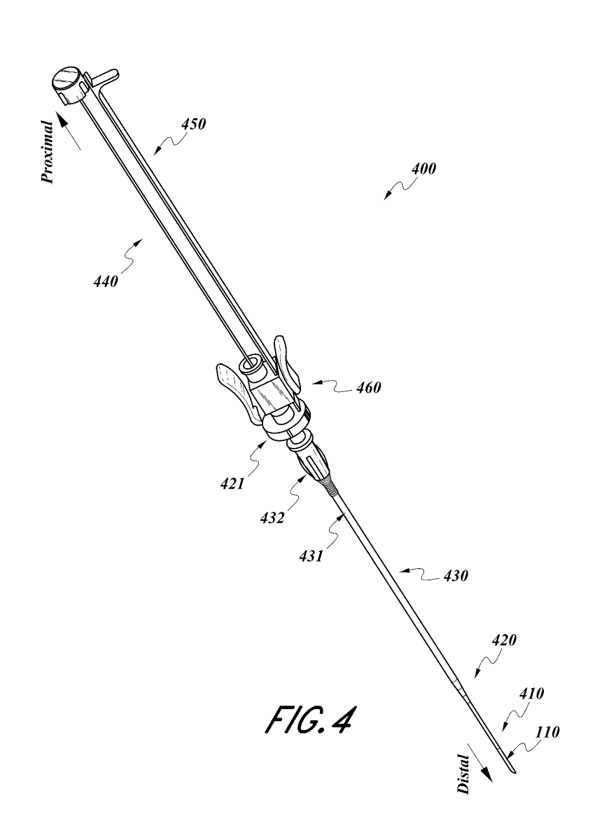

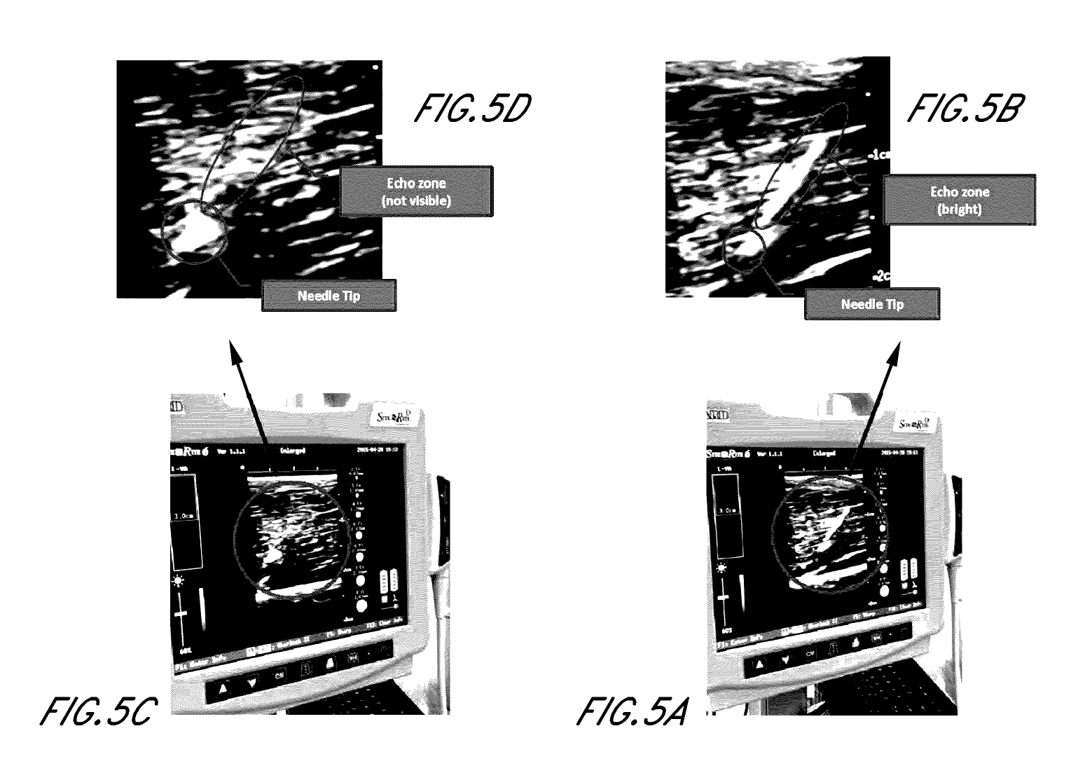

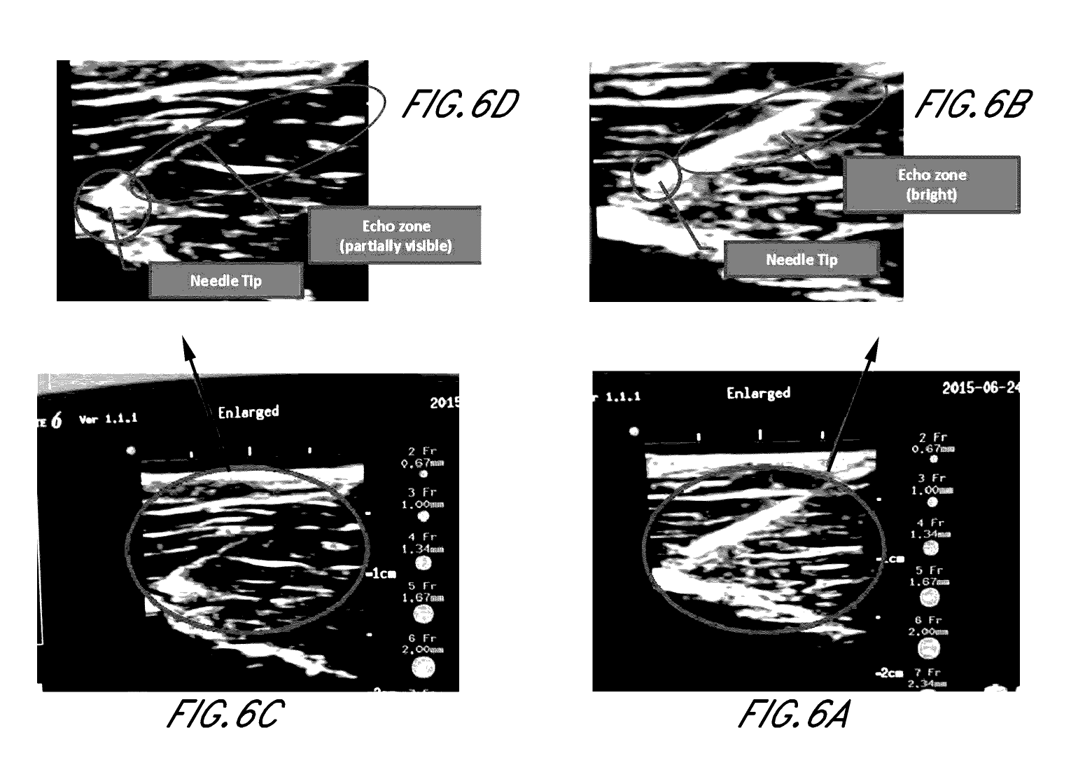

FIGS. 5 and 6 show comparative echo-photos of the medical article of FIGS. 1A-3B (as shown in FIGS. 5A, 5B, 6A, and 6B) and a needle having another echogenic design (as shown in FIGS. 5C, 5D, 6C, and 6D). In the illustrated embodiment, the first needle shown in FIGS. 5C, 5D, 6C, and 6D has a bead blasted echogenic portion or echo zone. The bead blasted echogenic portion has a roughened surface compared to the bare needle body. As shown, the bead blasted echogenic portion is not visible or has limited visibility under ultrasound. In contrast, the echogenic portion 120 of the medical article of FIGS. 1A-3B, shown in FIGS. 5A, 5B, 6A, and 6B, is significantly brighter on the ultrasound images.

In the illustrated embodiment, the echogenic portion 120 is located proximate to the distal end of the needle body 110 near the distal tip 200 to allow the physician to better visualize the location of the distal tip 200 during insertion of the needle into the patient's body. In other embodiments, the echogenic portion 120 may also or alternatively be positioned at other locations along the needle body 110 or extend over substantially the entire length of the needle body 110.

In some embodiments, the echogenicity of the needle body 110 can also be further increased by sandblasting the echogenic portion 120 and/or the distal tip 200 to roughen the needle surface. The distal tip 200 can be sharpened after sandblasting, allowing the distal tip 200 of the needle to be echogenic, as well. In some embodiments, the echogenic portion 120 and/or the distal tip 200 can comprise a material that scatters waves used in imaging, thus facilitating visualization of the needle under ultrasound. Echogenicity can also be increased by modifying the internal material of the needle itself, such as by adding granular impurities. However, in some instances modification of the internal material may unacceptably compromise the structural integrity of the needle. Advantageously, the echogenicity or similar imaging compatibility can allow an operator to easily view the distal tip 200 inside the body using a scanning technique such as ultrasound.

In the illustrated embodiment shown in FIG. 2, the distal tip 200 has a dual-beveled shape or configuration. In some embodiments, a first bevel 201 adjacent a distal end of the tip 200 comprises 40% to 60% of the total length of the dual-beveled tip 200 and is formed at an angle of 38° relative to a longitudinal axis of the needle. In some embodiments, a second bevel 202 adjacent a proximal end of the tip 200 comprises 40% to 60% of the total length of the dual-beveled tip 200 and is formed at an angle of 18° relative to a longitudinal axis of the needle.

In some embodiments, the needle body 110 can include at least one fenestration 300 or opening near the distal end of the needle body 110, as shown in FIGS. 3A-3B. The fenestration 300 extends, or provides a path, through the wall or side of the needle body 110. In some embodiments, the needle body 110 can be incorporated into a vascular access device 400, as described in greater detail herein. In such embodiments, the fenestration 300 can allow for a fluid, such as blood, to flow from inside the needle lumen to the outside of the needle during the use of the needle to be seen through a dilator or another object disposed around the needle. In some embodiments the fenestration 300 has a length of about 0.055 in. to about 0.065 in., for example 0.060 in., and is located about 4.475 in. to about 4.499 in., for example 4.487 in., from the proximal end of the needle. The fenestration 300 can have a variety of shapes and orientations on the needle body 110. For example, the fenestration 300 illustrated in FIG. 3B has an oblong shape. However, the shape of the side opening is not limited to the illustrated embodiment and may be round, square, or another shape. In some embodiments, the needle body 110 may have multiple side fenestrations or openings.

FIG. 4 illustrates an example embodiment of a medical access device 400 that can be inserted into a blood vessel (e.g., a vein or an artery) and that can include a needle body 110 according to the present disclosure. The access device 400 comprises a needle 410, a dilator 420, and a sheath 430. The dilator 420 includes a dilator shaft that extends distally from a dilator hub 421, and the sheath 430 includes a sheath body 431 that extends distally from a sheath hub 432. In the illustrated embodiment, the access device 400 also includes a guidewire section 440 and a track 450. The dilator 420 can be coaxially disposed about the needle 410, and the sheath 430 can be coaxially disposed about the dilator 420. The access device 400 can be configured to allow for telescoping movement among the needle 410, dilator 420, and sheath 430. The needle 410 includes needle body 110 and a needle hub 460 coupled to the proximal end of the needle body 110. In some embodiments, the needle body 110 is attached to the needle hub 460 via an attachment portion 130 (shown in FIGS. 1A and 3A) at or near the proximal end of the needle body 110. In the illustrated embodiment, the attachment portion 130 comprises a spiral pattern laser etched into the surface of the needle body 110. In other embodiments, a bead blast is applied to the attachment portion 130 to enhance bonding between the attachment portion 130 and the needle hub 460. Additional details regarding access devices that can incorporate a needle body 110 as described herein can be found in U.S. Publication No. 2014/0207069, the entirety of which is hereby incorporated by reference herein.

The sheath body 431 may be made partially or completely from clear, translucent, transparent, or semi-opaque material. The sheath body 431 may be a single piece sheath through which a catheter or other medical article (e.g., a guidewire 440) is inserted into the vessel. In such an embodiment, the sheath body 431 forms a conduit for insertion of the catheter or other medical article (e.g., a guidewire 440). The sheath hub 432 may include an engagement or locking structure that mates or engages the sheath hub 432 with a corresponding structure.

The sheath hub 432 preferably is designed so that a locking mechanism of the dilator hub 421 can enter the sheath hub 432 substantially unobstructed. However, in use, once the sheath hub 432 is placed at a desired location over the dilator shaft, the physician or healthcare provider can push, pull, or twist the sheath hub 432 and possibly disengage or engage the locking member with a corresponding connector on another medical article. The sheath hub 432 preferably comprises one or more surface features to allow the physician or healthcare provider to easily grasp or manipulate the sheath 430 and/or access device 400. For example, sheath hub 432 can include flatted portions to form, for example, a squared grip.

In use, the needle body 110 can be introduced into a target location in the patient's body, for example, under ultrasound guidance. The guidewire section 440 can then be inserted into the target location by advancing the guidewire section 440 distally relative to the needle body 110. The dilator 420 and sheath 430 can then be inserted into the target location by advancing the dilator 420 and sheath 430 distally relative to the needle body 110. The dilator 420, needle body 110, and guidewire section 440 can then be removed from the patient's body, leaving the sheath 430 in place. In some cases, if the needle body 110 includes an echogenic portion with grooves that form, for example, a spiral pattern, a ring or series of rings, or another pattern including grooves extending generally perpendicularly to the longitudinal axis of the needle body 110, a distal edge of the dilator 420 could catch or snag in the grooves. This could inhibit the dilator 420 from smoothly sliding over the needle body 110 into the target location. The physician may therefore need to apply greater force to the dilator 420 to advance the dilator 420 over the needle body 110, which could increase the risk of the distal tip 200 of the needle body 110 being advanced too far into the patient's body or other adverse effects. In some cases, the dilator 420 may be damaged if the distal edge catches or snags in one or more grooves. However, if the needle body 110 has an echogenic portion 120 including one or more grooves 121 formed in a zigzag pattern, as shown in FIGS. 1A-2, or another pattern with grooves extending generally more parallel to the longitudinal axis of the needle body 110, the distal edge of the dilator 420 is advantageously less likely to be caught in or snagged by the grooves.

The needle body 110 can be made of any of a variety of materials known in the art. The embodiments herein described are comprised of conventional, biocompatible materials. For example, the needle body 110 preferably consists of ceramic, a rigid polymer, or a metal such as stainless steel, nitinol, or the like. In some embodiments, one or more portions of needle body 100 can comprise a material, or can include a treatment or surface coating of a material, such as surfactants, lubricious coatings, and/or coatings with desired hydrophilic and/or hydrophobic properties, to provide additional functionality to needle.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

What is claimed is:

1. A medical article comprising:
a needle body having a proximal end, a distal end, and a lumen extending therethrough, the needle body comprising an echogenic portion, the echogenic portion comprising at least one groove having a zigzag configuration.

2. The medical article of claim 1, wherein the zigzag configuration circumferentially encircles at least a portion of the needle body proximate to the distal end, and wherein the at least one groove is configured to increase echogenic properties of the needle body.

3. The medical article of claim 1, wherein the at least one groove encircles an entire circumference of the needle body.

4. The medical article of claim 1, wherein the echogenic portion comprises a plurality of grooves, each of the plurality of grooves having the zigzag configuration and encircling the needle body.

5. The medical article of claim 4, wherein each of the plurality of grooves comprises a series of alternating proximal facing peaks and distal facing peaks, and wherein the proximal facing peaks of a first of the plurality of grooves are at least substantially circumferentially aligned with the distal facing peaks of a second of the plurality of grooves positioned adjacent and proximal to the first of the plurality of grooves.

6. The medical article of claim 1, wherein the at least one groove has a width of about 0.0029″.

7. The medical article of claim 1, wherein the distal end comprises a beveled tip.

8. The medical article of claim 1, further comprising at least one opening in a wall of the needle body.

9. The medical article of claim 1, wherein the proximal end comprises a laser-etched right-hand spiral configured to attach to a needle hub.

10. A medical article comprising:
a needle body having a proximal end, a distal end, and a lumen extending therethrough, the distal end comprising an echogenic portion having a plurality of grooves, each of the plurality of grooves comprises a series of alternating proximal facing peaks and distal facing peaks.

11. The medical article of claim 10, wherein adjacent grooves are spaced apart by about 0.031″.

12. The medical article of claim 10, wherein the plurality of grooves encircle an entire circumference of the needle.

13. The medical article of claim 10, wherein the grooves have a depth of about 0.0022″.

14. The medical article of claim 10, wherein the proximal facing peaks of a first of the plurality of grooves are at least substantially circumferentially aligned with the distal facing peaks of a second of the plurality of grooves positioned adjacent and proximal to the first of the plurality of grooves.

15. An access device comprising:
a needle having an elongated needle body and a needle hub from which the needle body extends, the needle body comprising a proximal end, a distal end, a lumen extending therethrough, and an echogenic portion, the echogenic portion being formed by at least one groove shaped in a zigzag configuration;
a dilator slideably disposed on the needle body, the dilator comprising a dilator hub and an elongated dilator shaft that extends from the dilator hub; and
a sheath slideably disposed on the dilator shaft, the sheath comprising a tubular sheath body and a sheath hub coupled with the tubular sheath body.

16. The access device of claim 15, wherein the zigzag configuration circumferentially encircles at least a portion of the needle body proximate to the distal end, and wherein the at least one groove is sized and shaped to increase echogenic properties of the needle body.

17. The access device of claim 15, wherein the at least one groove encircles an entire circumference of the needle.

18. A method of forming an echogenic needle body, the method comprising:
forming grooves in a surface of the needle body; and
grinding a distal end of the needle body to form a beveled tip.

19. The method of claim 18, wherein forming grooves in the surface of the needle body comprises laser etching the grooves in a zigzag pattern circumferentially encircling the needle body.

20. A method of using a medical article comprising at least a single groove in a zigzag pattern encircling a needle body proximate to a distal end, the method comprising:
inserting the medical article into a vessel of a patient;
placing an ultrasound transducer in proximity to the patient and above the medical article;
transmitting ultrasound energy in a direction towards the zigzag pattern on the medical article; and
sensing a return signal reflected off the zigzag pattern to ensure proper insertion of the medical article into the vessel.
